(19) United States
(12) Patent Application Publication
(10) Pub. No.: US 2018/0064409 A1
(43) Pub. Date: Mar. 8, 2018

(54) SIMULTANEOUSLY DISPLAYING MEDICAL IMAGES

(71) Applicant: KONINKLIJKE PHILIPS N.V., Eindhoven (NL)

(72) Inventors: Alexander Schmidt-Richberg, Hamburg (DE); Tobias Klinder, Uelzen (DE); Sven Kabus, Hamburg (DE); Rafael Wiemker, Kisdorf (DE)

(21) Appl. No.: 15/691,876

(22) Filed: Aug. 31, 2017

(30) Foreign Application Priority Data
Sep. 5, 2016 (EP) 16187235.3

(51) Int. Cl. A61B 6/00; A61B 8/08; G06F 19/00

(52) U.S. Cl. CPC A61B 6/463; A61B 6/5229; A61B 5/055; G06F 19/321; A61B 8/5223

(57) ABSTRACT

A system and method are provided for displaying medical images. A first viewport is generated which shows a part of a first medical image which shows a region of interest. A second viewport is generated which shows a part of a second medical image which shows a corresponding region of interest, e.g., representing a same anatomical structure or a same type of anatomical structure. In order to establish this ‘synchronized’ display of regions of interest, a displacement field is estimated between the first medical image and the second medical image. However, the displacement field is not used to deform the second medical image. Rather, the displacement field is used to identify the corresponding region of interest and thereby which part of the second medical image is to be shown. It is thus avoided that the second medical image itself is deformed, which would typically also deform the region of interest and thereby impair its assessment.

### FIELD OF THE INVENTION

The invention relates to a system and a method for displaying medical images. The invention further relates to a server, imaging apparatus and workstation comprising the system. The invention further relates to a computer readable medium comprising instructions to cause a processor system to perform the method.

### BACKGROUND OF THE INVENTION

Medical images may show one or more anatomical structures of a patient and/or functional properties of underlying tissue, with such tissue being in the following also considered as an example of an anatomical structure. It may be desirable to determine changes in (part of) an anatomical structure. Such changes may represent a change in disease state or other type of anatomical change. For example, a change may be due to, or associated with, growth of a tumor, progression of Multiple Sclerosis (MS), etc. A specific example is that in the field of pulmonary image analysis, such changes may relate to the size or shape of pathologies, such as lung nodules, tumors or fibrosis. By determining the change and the type of change, it may be possible to better treat the disease, e.g., by adjusting treatment strategy.

For the detection of such changes, two or more medical images may be compared which show the anatomical structure at different moments in time. Such medical images are also referred to as longitudinal images, and the changes are also known as longitudinal changes. Alternatively or additionally, two or more medical images may differ in other aspects, e.g., relating to a healthy patient and a diseased patient, etc.

A common approach for enabling the determining of such changes, or in general the differences between medical images, is to display the medical images simultaneously, e.g., side by side in respective viewports of a graphical user interface.

However, due to various reasons, anatomical structures may be differently aligned in such medical images. This may caused by, e.g., varying positions of a patient in an imaging apparatus during subsequent scans, different breathing states during image acquisition, or in the case of the medical images of being of different patients, the anatomy of the patients being different. Such differences in alignment may hinder the interpretation of the medical images, as it may require the user, e.g., a clinician such as a radiologist, to mentally match the anatomical structures across the different medical images.

It is commonly known to employ image registration techniques to establish anatomical correspondences between medical images. Such image registration typically involves determining a transformation between the medical images, e.g., a linear or non-linear transformation, and then applying the transformation, e.g., by translating, rotating and/or deforming one or more of the medical images in accordance with the transformations. Linear transformations are global in nature and thus cannot model local geometric differences between medical images. Non-linear transformations, which are also known as ‘elastic’ or ‘nonrigid’ transformations, are able to cope with local differences between medical images, and thus are able to better align the anatomical structures across different medical images.

### SUMMARY OF THE INVENTION

The inventors have recognized that linear transformations establish insufficient alignment between medical images. For example, when simultaneously zooming into the medical images, the respective viewports may show different and/or unrelated anatomical structures. Non-linear registration addresses this problem, but it may locally deform the image content and thereby also deform pathologies shown in the medical images. Disadvantageously, the assessment of changes is impaired when using non-linear registration.

It would be advantageous to obtain a system and method for displaying medical images which addresses one or more of the above problems.

A first aspect of the invention provides a system for displaying medical images, comprising:
an image data interface configured to access image data of a first medical image and a second medical image;
a memory comprising instruction data representing a set of instructions;
a processor configured to communicate with the image data interface and
the memory and to execute the set of instructions, wherein the set of instructions, when executed by the processor, configure the processor to:
   receive selection data indicative of a region of interest in the first medical image;
   generate display data comprising a first viewport, the first viewport comprising a part of the first medical image which shows the region of interest;
   identify a corresponding region of interest in the second medical image; and
   generate the display data to additionally comprise a second viewport, the second viewport comprising a part of the second medical image which shows the corresponding region of interest;
wherein the set of instructions, when executed by the processor, configure the processor to identify the corresponding region of interest in the second medical image by:
estimating a displacement field by performing a non-linear registration between the first medical image and the second medical image; and
identifying the corresponding region of interest using one or more displacement vectors of the displacement field which match the region of interest in the first medical image to the corresponding region of interest in the second medical image.

A further aspect of the invention provides a server, workstation or imaging apparatus comprising the system.

A further aspect of the invention provides a method of displaying medical images, comprising:
accessing a database comprising a first medical image and a second medical image;
receiving selection data indicative of a region of interest in the first medical image;
generating display data comprising a first viewport, the first viewport comprising a part of the first medical image which shows the region of interest;
identifying a corresponding region of interest in the second medical image; and
generating the display data to additionally comprise a second viewport, the second viewport comprising a part of the second medical image which shows the corresponding region of interest;
wherein the identifying the corresponding region of interest in the second medical image comprises:
estimating a displacement field by performing a non-linear registration between the first medical image and the second medical image; and
identifying the corresponding region of interest using one or more displacement vectors of the displacement field which match the region of interest in the first medical image to the corresponding region of interest in the second medical image.

A further aspect of the invention provides a computer readable medium comprising transitory or non-transitory data representing instructions to configure a processor system to perform the method.

The above measures provide an image data interface configured to access image data of a first medical image and a second medical image. A non-limiting example is that the medical images may be accessed from an image repository, such as a Picture Archiving and Picture Archiving and Communication System (PACS).

The above measures further provide a processor configured by way of a set of instructions to receive selection data indicative of a region of interest in the first medical image. For example, the region of interest may represent an anatomical structure such as a blood vessel, nodule, lesion or airway, which may be selected by a user, or automatically detected by the system, or in another manner indicated to the processor. It is noted that the region of interest may represent a segmentation of the anatomical structure, e.g., providing a pixel- or voxel-accurate delineation. Alternatively, the region of interest may include but not directly delineate the anatomical structure, e.g., by being constituted by a bounding box which includes the anatomical structure and its immediate surroundings.

The processor may then identify a corresponding region of interest in the second medical image by making use of a displacement field which is obtained by non-linear registration between the first medical image and the second medical image. The displacement field may be estimated by the processor to identify the corresponding region of interest, or may have been estimated previously, e.g., when identifying another corresponding region of interest. The displacement field may be represented by a vector field, and in general may also be known in the art as a ‘dense’ displacement field. It is noted that such types of displacement fields, and their estimation, is known per se from the field of image registration, and as well as from neighboring fields such as motion estimation.

The processor may then identify the corresponding region of interest as a function of the displacement field, and in particular, using one or more displacement vectors of the displacement field which match the region of interest in the first medical image to the corresponding region of interest in the second medical image. A non-limiting example is that a displacement vector may be selected which represents the displacement of a center of the region of interest. The displacement vector may thereby be indicative of the relative position of the center of the corresponding region of interest in the second medical image. As such, the coordinates of the corresponding region of interest may be obtained by adding the vector components to the coordinates of the first region of interest.

The processor may then generate display data which comprises a first viewport and a second viewport. The first viewport comprises a part of the first medical image which shows the region of interest, and the second viewport comprises a part of the second medical image which shows the corresponding region of interest. For example, each respective part may be a rectangular part from the respective medical image, which may include the respective region of interest and its immediate neighborhood, e.g., by being shaped as a bounding box. Alternatively, the region of interest may itself represent a bounding box, and each respective part may simply correspond to the region of interest.

The above measures have as effect that two viewports are provided which show selected parts of the respective medical images. Both viewports are ‘synchronized’ in that they show a corresponding region of interest, such as a same (type of) anatomical structure, rather than simply showing a same position in each medical image. To compensate for a possible misalignment of the region of interests across the medical images, a displacement field is estimated and subsequently used to link the region of interest in the first medical image, which is shown in the first viewport, is to a corresponding region of interest in the second medical image, which is then shown in the second viewport.

As such, rather than deforming the second medical image using the displacement field, the displacement field is only used to identify a part of the second medical image which corresponds to a selected part of the first medical image, which is then displayed. Effectively, if both medical images have a same spatial coordinate system, the coordinates of the second viewport may be obtained by adding a displacement vector representing its displacement to the coordinates of the first viewport. The second viewport may thus have an ‘image offset’ with respect to the first viewport in this coordinate system, which may represent a translation. It is thus avoided that the second medical image is deformed, which may also deform the region of interest and thereby impair its assessment.

It will be appreciated that by showing only said parts in said viewports, a zoomed-in view of the respective medical images may be provided, compared to the situation in which the entire medical images were to be displayed in each respective viewport. As such, each viewport may provide a zoomed-in view of the respective medical image, with both zoomed-in views being ‘synchronized’ in that they show a same (type of) region of interest, rather than simply showing a co-located part of the respective medical image.

Optionally, the set of instructions, when executed by the processor, configure the processor to apply a spatial interpolation to the displacement field to determine the one or more displacement vectors which match the region of interest in the first medical image to the corresponding region of interest in the second medical image. By using a spatial interpolation, the displacement field may be estimated and/or stored in a memory at a lower resolution than may otherwise be needed for use by the system.

Optionally, the set of instructions, when executed by the processor, configure the processor to estimate or convert the displacement field in a format having at least one of:
a vector precision limited to integer precision; and
a spatial resolution which is lower than the spatial resolution of the first medical image and/or the second medical image.

The inventors have recognized that the claimed use of the displacement field is less critical in terms of vector accuracy than the conventional use of deforming a medical image. Namely, the displacement vectors may be ‘merely’ used to determine an image offset for the second viewport. It has been found that for such use, the vectors may be relatively coarse, e.g., at integer precision rather than having sub-pixel or sub-voxel precision. Likewise, the spatial resolution, and thus spatial accuracy, may be lower than, e.g., the spatial resolution of the first medical image and/or the second medical image, and rather be interpolated ‘on the fly’ during use, e.g., using a zero, first or higher order spatial interpolation. As such, the computational complexity of estimating the displacement field, and the storage requirements of storing the displacement field, may be reduced.

Optionally, the set of instructions, when executed by the processor, configure the processor to re-use the displacement field to identify another corresponding region of interest in the second medical image in response to subsequently received selection data which is indicative of another region of interest in the first medical image. The displacement field may be estimated once for a pair of medical images, rather than being estimated for each newly selected region of interest in the first medical image. As such, the computational complexity of identifying the corresponding region of interest may be reduced.

Optionally, the system may further comprise a user input interface connectable to a user input device operable by a user, wherein the selection data represents a selection of the region of interest using the user input device. As such, the user may manually select the region of interest in the first medical image, e.g., using an onscreen pointer.

Optionally, the set of instructions, when executed by the processor, configure the processor to generate the display data to additionally comprise a further viewport which shows the first medical image, and wherein the selection data represents a selection of the region of interest in the third viewport using an onscreen pointer controllable by the user input device. As such, in addition to showing a part of the first medical image in a first viewport, the system may be configured to display the first medical image in substantially its entirety in a further viewport. This further viewport may thus provide a global overview of the first medical image in which the region of interest may be selected by the user, with the first viewport then providing a zoomed-in view of the selected region of interest.

Optionally, the set of instructions, when executed by the processor, configure the processor to generate the display data to additionally comprise a further viewport which shows a list of regions of interest comprised in the first medical image, and wherein the selection data represents a selection of the region of interest from said list. If a list of regions of interest is available, e.g., as detected by a Computer Aided Detection (CAD) algorithm, the region of interests may be displayed in a list to enable the user to select one of the regions of interest for being shown in the first viewport. It is noted that such CAD algorithms and similar algorithms are known per se in the art of medical image analysis. The set of instructions may include a subset of instructions which represent said algorithm.

Optionally, the set of instructions, when executed by the processor, configure the processor to:
estimate a rotation between the first medical image and the second medical image from the displacement field; and
rotate the part of the second medical image to compensate for the rotation before showing said part in the second viewport.

Rather than only calculating a translational image offset for the second viewport, the processor may also calculate a rotational image offset, namely by estimating a rotation between the first medical image and the second medical image from the displacement field. It is noted that such rotation also does not deform the image between the first medical image and the second medical image, as it may rather represents a global or regional rotation. For example, the rotation may be estimated by estimating an affine transformation representing the rotation from the displacement field.

Optionally, the set of instructions, when executed by the processor, configure the processor to estimate the rotation from the displacement field in, or in a neighborhood of, a region of the displacement field which corresponds to the region of interest in the first medical image. The rotation is thus specifically estimated for the region of interest, or for a neighborhood which includes the region of interest. The neighborhood may correspond to the part of the first medical image which is shown in the first viewport. The rotation may represent the curl or rotor of the displacement field in said neighborhood, and may be calculated in a manner known per se from the field of vector calculus.

In accordance with the abstract of the present disclosure, a system and method may be provided for displaying medical images. A first viewport may be generated which shows a part of a first medical image which shows a region of interest. A second viewport may be generated which shows a part of a second medical image which shows a corresponding region of interest, e.g., representing a same anatomical structure or a same type of anatomical structure. In order to establish this ‘synchronized’ display of regions of interest, a displacement field may be estimated between the first medical image and the second medical image. However, the displacement field is not used to deform the second medical image. Rather, the displacement field may be used to identify the corresponding region of interest and thereby which part of the second medical image is to be shown. It may thus be avoided that the second medical image itself is deformed, which would typically also deform the region of interest and thereby impair its assessment.

It will be appreciated by those skilled in the art that two or more of the above-mentioned embodiments, implementations, and/or optional aspects of the invention may be combined in any way deemed useful.

Modifications and variations of the server, the workstation, the imaging apparatus, the method, and/or the computer program product, which correspond to the described modifications and variations of the system, can be carried out by a person skilled in the art on the basis of the present description.

A person skilled in the art will appreciate that the system and method may be applied to multi-dimensional image data, e.g., two-dimensional (2D), three-dimensional (3D) or four-dimensional (4D) images, acquired by various acquisition modalities such as, but not limited to, standard X-ray Imaging, Computed Tomography (CT), Magnetic Resonance Imaging (MRI), Ultrasound (US), Positron Emission Tomography (PET), Single Photon Emission Computed Tomography (SPECT), and Nuclear Medicine (NM).

The image data may be longitudinal image data, including but not limited to longitudinal image data obtained for lung cancer screening in CT scans, progression assessment of dementia in MR images, or monitoring the success of various treatments.

### BRIEF DESCRIPTION OF THE DRAWINGS

These and other aspects of the invention will be apparent from and elucidated further with reference to the embodiments described by way of example in the following description and with reference to the accompanying drawings, in which

FIG. 1 shows a system for displaying medical images;

FIG. 2A shows a part of a first medical image which is to be displayed in a viewport, thereby providing a zoomed-in view of the first medical image;

FIG. 2B shows a displacement field representing the local displacements between the first medical image and a second medical image;

FIG. 2C shows a part of a second medical image which corresponds to the part of the first medical image having been identified using the displacement field;

FIG. 3A shows an example of a part of a first medical image;

FIG. 3B shows an example of a corresponding part of a second medical image which was identified using the displacement field;

FIG. 3C shows an example of a part of the second medical image after being deformed in accordance with the displacement field;

FIG. 4 shows a graphical user interface comprising a viewport in which a user may select a region of interest in a medical image, a number of viewports providing zoomed-in views of the region of interest and corresponding regions of interests in other medical images, and a viewport providing information on the selected region of interest;

FIG. 5 shows a method for displaying medical images; and

FIG. 6 shows a computer readable medium comprising instructions for causing a processor system to perform the method.

It should be noted that the figures are purely diagrammatic and not drawn to scale. In the Figures, elements which correspond to elements already described may have the same reference numerals.

### LIST OF REFERENCE NUMBERS

The following list of reference numbers is provided for facilitating the interpretation of the drawings and shall not be construed as limiting the claims.

020 image repository

022 first medical image

024 second medical image

040 user input device

042 user input data

062 display data

080 display

100 system for displaying medical images

120 image data interface

122 data communication

130 memory

132 data communication

140 user input interface

142 data communication

160 processor

200 part of first medical image comprising region of interest

202 part of second medical image comprising corresponding region of interest

210 co-located part of non-linearly registered second medical image

220, 222, 224 lesion

230 displacement field

232 displacement vector

300 graphical user interface

310 viewport showing zoomed-in view of first medical image

312 viewport showing zoomed-in view of second medical image

314 viewport showing zoomed-in view of third medical image

320 viewport showing global view of third medical image

330 part of third medical image comprising region of interest

340 viewport showing information on region of interest

400 method for displaying medical images

410 accessing medical images

420 receiving selection data indicative of region of interest

430 identifying corresponding region of interest

440 estimating displacement field

450 identify corresponding region of interest using displacement vector(s)

460 generating output image

500 computer readable medium

510 instructions stored as non-transient data

### DETAILED DESCRIPTION OF EMBODIMENTS

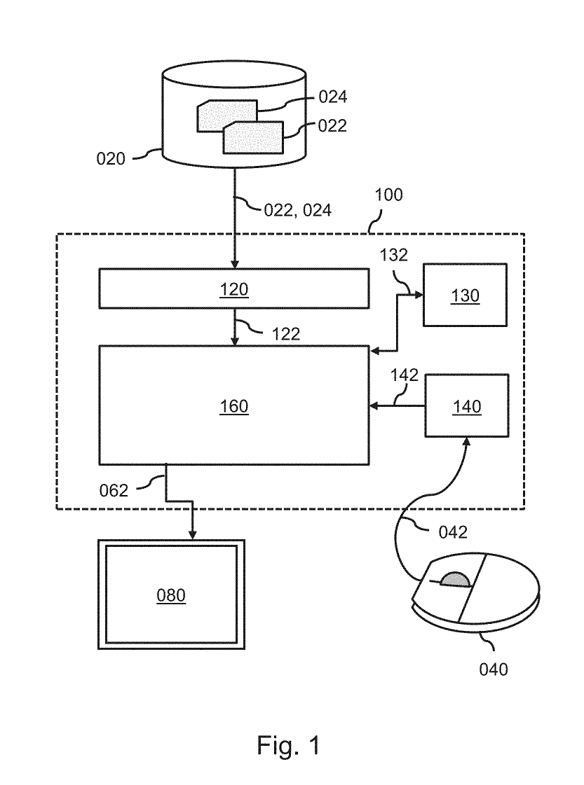

FIG. 1 shows a system 100 which is configured for displaying medical images. The system 100 comprises an image data interface 120 configured to access a first medical image and a second medical image. In the example of FIG. 1, the image data interface 120 is shown to be connected to an external image repository 020 which comprises the image data of the first medical image 022 and the second medical image 024. For example, the image repository 020 may be constituted by, or be part of, a Picture Archiving and Communication System (PACS) of a Hospital Information System (HIS) to which the system 100 may be connected or comprised in. Accordingly, the system 100 may obtain access to the image data of the first medical image 022 and the second medical image 024 via the HIS. Alternatively, the image data of the first medical image 022 and the second medical image 024 may be accessed from an internal data storage of the system 100. In general, the image data interface 120 may take various forms, such as a network interface to a local or wide area network, e.g., the Internet, a storage interface to an internal or external data storage, etc.

The system 100 further comprises a processor 160 configured to internally communicate with the image data interface 120 via data communication 122, as well as a memory 130 accessible by the processor 140 via data communication 132. The memory 130 may comprise instruction data representing a set of instructions which configures the processor 160 to, during operation of the system 100, receive selection data indicative of a region of interest in the first medical image 022, generate display data 062 comprising a first viewport, with the first viewport comprising a part of the first medical image which shows the region of interest, identify a corresponding region of interest in the second medical image 024, and generate the display data 062 to additionally comprise a second viewport, the second viewport comprising a part of the second medical image which shows the corresponding region of interest. In this respect, it is noted that the generating of the display data 062 to comprise the first viewport and the second viewport may be performed together, e.g., in one operation, even though it may be described as individual operations elsewhere.

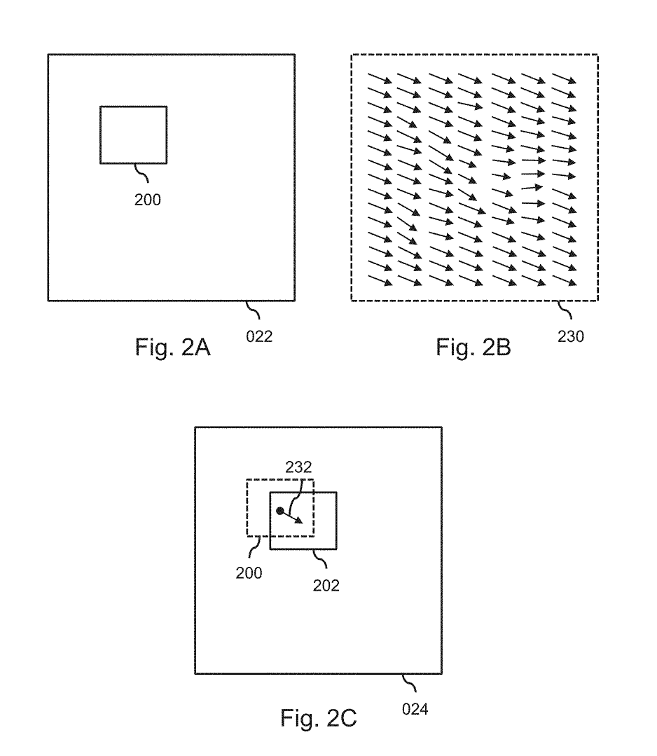

Moreover, the set of instructions, when executed by the processor 160, may configure the processor 160 to identify the corresponding region of interest in the second medical image by estimating a displacement field by performing a non-linear registration between the first medical image and the second medical image, and identifying the corresponding region of interest using one or more displacement vectors of the displacement field which match the region of interest in the first medical image to the corresponding region of interest in the second medical image. The displacement field may in the following also be referred to as a ‘dense’ displacement field. These and other aspects of the operation of the system 100 will be further elucidated with reference to FIGS. 2A-4.

FIG. 1 further shows an optional aspect of the system 100, in that the processor 160 may be configured to directly output the display data 062 to an external display 080. Alternatively, the display may be part of the system 100. Alternatively, the display data 062 may be output to the display 080 by a separate display output (not shown in FIG. 1).

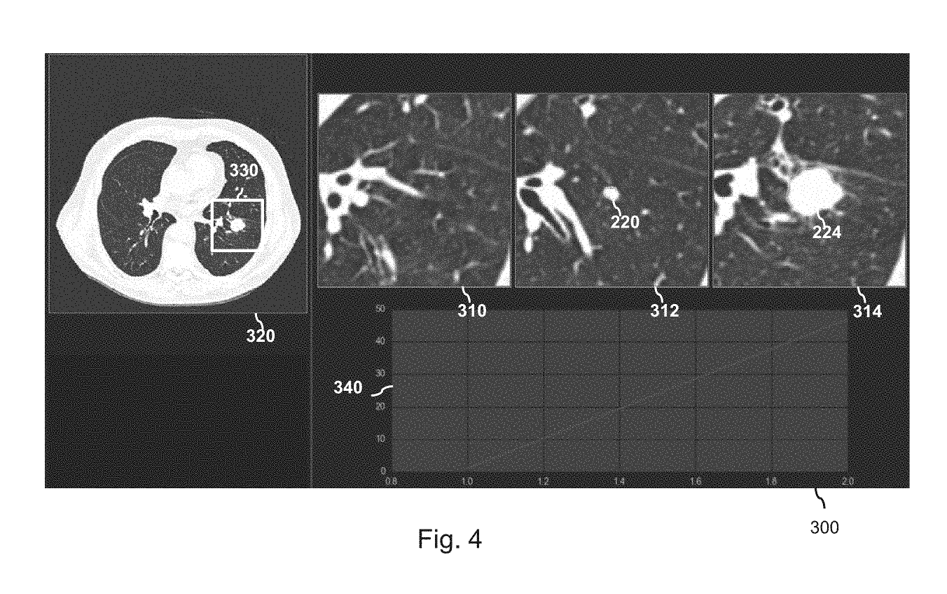

FIG. 1 further shows that the system 100 may optionally comprise a user input interface 140 which may be configured to enable a user to select the region of interest via a user input device 040, e.g., on the basis of user input data 042 generated by the user input device 040. This functionality will be further explained with reference to FIG. 4. The user input device 040 may take various forms, including but not limited to a computer mouse, touch screen, keyboard, etc. FIG. 1 shows the user input device to be a computer mouse 040. In general, the user input interface 140 may be of a type which corresponds to the type of user input device 040, i.e., it may be a thereto corresponding user device interface.

The system 100 may be embodied as, or in, a device or apparatus, such as a server, workstation, imaging apparatus or mobile device. The device or apparatus may comprise one or more microprocessors or computer processors which execute appropriate software. The processor of the system may be embodied by one or more of these processors. The software may have been downloaded and/or stored in a corresponding memory, e.g., a volatile memory such as RAM or a non-volatile memory such as Flash. The software may comprise instructions configuring the one or more processors to perform the functions described with reference to the processor of the system. Alternatively, the functional units of the system, e.g., the image data interface, the user input interface and the processor, may be implemented in the device or apparatus in the form of programmable logic, e.g., as a Field-Programmable Gate Array (FPGA). The image data interface and the optional user input interface may be implemented by respective interfaces of the device or apparatus. In general, each functional unit of the system may be implemented in the form of a circuit. It is noted that the system 100 may also be implemented in a distributed manner, e.g., involving different devices or apparatuses. For example, the distribution may be in accordance with a client-server model, e.g., using a server and a thin-client PACS workstation.

FIG. 2A schematically shows a first medical image 022 in the form of an outline of said image, with a part 200 in the first medical image being indicated which comprises a region of interest (not shown). The part 200, or the region of interest contained therein, may have been selected by the user, e.g., as described with reference to FIG. 4. The selection may be for the purpose of said part being displayed in a separate viewport, e.g., to provide a zoomed-in view of the first medical image 022. Alternatively, the part 200 may have been selected automatically by the system, or may have been selected in another manner. A non-limiting example is that the region of interest may have been segmented by the system, with the part 200 representing a bounding box centered on said segmentation. It will be appreciated that the segmentation of a region of interest is well from the field of medical image analysis, and may be based on, e.g., a user-selected seed point.

FIG. 2B shows a dense displacement field 230 representing the local displacements between the first medical image 022 of FIG. 2A and a second medical image (shown in FIG. 2C). Both medical images may relate to each other, e.g., by representing longitudinal image data of a same patient, by showing a similar anatomical structure of different patients, etc. As such, the dense displacement field 230 may establish a registration between both medical images, allowing the second medical image to be deformed to match the first medical image, or vice versa. Although previously explained with respect to a second medical image, there may be multiple target images I₁(x), . . . , Iₙ₋₁(x) which may be registered to a reference image I₀ (x) in the form of the first medical image. This may be done using any non-linear registration algorithm, e.g., a fast elastic image registration as described in the (workshop) paper “Fast elastic image registration” by Kabus et al., Medical Image Analysis for the Clinic: A Grand Challenge, MICCAI 2010. This may result in a dense displacement field 230 u₁(x), . . . , uₙ₋₁(x) for each target image, with each dense displacement field indicating for each coordinate x of the reference image the corresponding coordinate x+uₜ(x) in the respective target image Iₜ (x). For each of these target images, a viewport may be generated in the manner as described below with reference to FIG. 2C.

In this respect, it is noted that FIG. 2B shows a two-dimensional vector field 230. However, that if the medical images have a different dimensionality, e.g., three-dimensional, the dense displacement field(s) may have a same dimensionality. Alternatively, the dense displacement field(s) may have a lower dimensionality, e.g., when one of the dimensions of the medical images relates to a time dimension rather than spatial dimension.

FIG. 2C shows a part 202 of the second medical image 024 which comprises a region of interest (not shown explicitly) which corresponds to the region of interest comprised in the part 200 of the first medical image of FIG. 2A, with said corresponding region of interest, and thereby the part 202, having been identified using the dense displacement field of FIG. 2B. Namely, a coordinate x associated with the region of interest of FIG. 2A, e.g., being the center point of the part 200, may be translated by a vector 232 uₜ(x) to identify a coordinate x+uₜ(x) which is associated with the region of interest in the second medical image 024, e.g., thereby yielding a center point of the part 202.

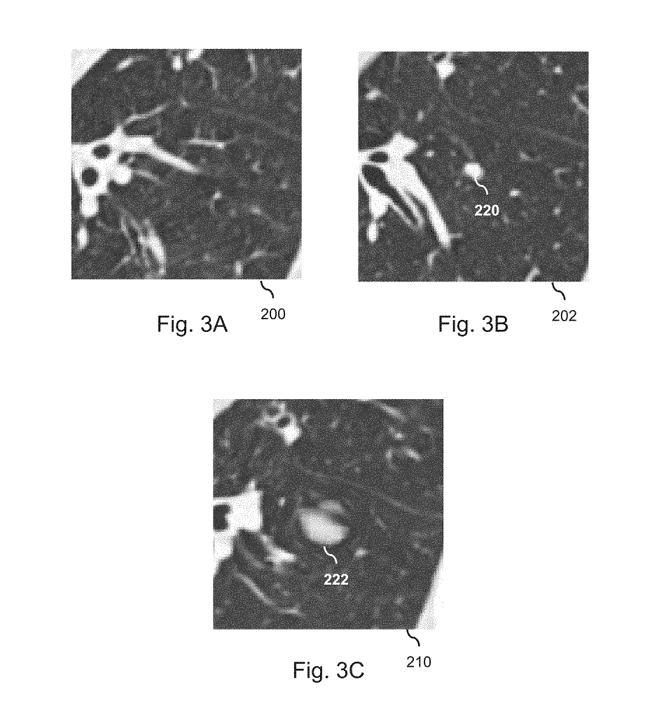

FIGS. 3A-3B show corresponding parts 200, 202 of a first medical image and a second medical image, respectively, which have been identified using such a dense displacement field, e.g., in the manner as described with reference to FIGS. 2A-2C. FIG. 3B is shown to comprise a lesion 220 which may have appeared in the time between acquisition of the first medical image and the second medical image. It will be appreciated that the lesion 220 is shown ‘as-is’, e.g., without being deformed by non-linear image registration, as the part 202 is simply a ‘cut-out’ of the second medical image, e.g., a selection of image data. FIG. 3C rather shows the second medical image being deformed in accordance with the dense displacement field, thereby obtaining a ‘warped’ second medical image in which a part 210 which is co-located with the part 200 of FIG. 3A, e.g., having same or corresponding coordinates, directly shows the lesion 222, but with the lesion being deformed. Disadvantageously, the display of the part 210 may be unsuitable for assessing the lesion 222, and/or the change in the lesion 222 with respect to the first medical image.

FIG. 4 shows a graphical user interface 300 which may be generated by the system of FIG. 1 and displayed on a display to a user. The graphical user interface 300 is shown to comprise a viewport 320 which may also be referred to as ‘global view’ in the following. The global view 320 may show a medical image representing a whole reference scan, e.g., a most recent examination of a patient. Such a medical image is henceforth also referred to as ‘scan’. The global view 320 may enable a global assessment of the patient's anatomy. Moreover, the global view 320 may be used by the user to select a region of interest. For example, the user may operate a user input device to position an onscreen pointer (not shown in FIG. 4), and select the region of interest using the onscreen pointer, e.g., by drawing a bounding box 330 in the reference scan shown in the global view 320.

In response to the selection of the region of interest, a viewport 314 may provide a zoomed-in view of the medical image shown in the global view 320, with the zoomed-in view showing the image data in the bounding box 330. Such a zoomed-in view may in the following also be referred to as ‘focus view’. As also shown in FIG. 4, a number of n focus views may be displayed simultaneously, where n is the number of available or selected examinations, being in this case 3 focus views 310-314 including the focus view 314 of the reference scan. The additional focus views 310, 312 may show the same anatomical position in a different scan, with the view shown by the focus view being determined by registering each scan to the reference scan, e.g., in the manner described with reference to FIGS. 2A-2C, and translating each scan within its viewport 310, 312. As such, the scans are not deformed but rather translated, and possibly rotated, to the correct position within each viewport. Effectively, the non-linear transformation may be locally approximated by a translation, and possibly a rotation. This may avoid distortions of the image content of each scan. It is noted that the zoom factor in the focus views 310-314 may be a fixed value, or may be chosen according to the content of the region of interest, e.g., the tumor size, etc.

Although not shown in FIG. 4, the graphical user interface 300 may optionally comprise a viewport comprising a list of regions of interest. This may enable quick navigation between the different regions of interest, e.g., by not requiring the user to manually select a region of interest from the global view 320. For example, a list of pre-determined regions of interest may be provided. In a specific example, this list may comprise potential lung nodules which may have been determined by an automatic CAD algorithm, e.g., as described in “Pulmonary nodule detection using a cascaded SVM classifier” by Bergtholdt et al., Proc. SPIE 9785, Medical Imaging 2016: Computer-Aided Diagnosis, March 2016. A selection of a list element may automatically focus each focus view on the selected region of interest in each of the focus views, or on an anatomically corresponding position, e.g., if one of the scans does not comprise the selected long nodule. Additionally or alternatively, the graphical user interface 300 may comprise a viewport 340 showing information on the selected region of interest. For example, geometric or functional parameters of the structure contained in the region of interest may be displayed. In a specific example, the change of volume or diameter of a lung nodule over time may be displayed.

In general, the selection of the region of interest may be performed in various ways, including but not limited to: selecting one of a set of pre-determined regions of interest, e.g., by browsing through a list of regions of interest, or by selecting a segmentation outline of the region of interest in the global view 320. Another option is that the user may select a specific position in the global view 320, or may continuously select different positions, e.g., by moving the mouse over the reference scan with the mouse button depressed. In response, the focus views may show a zoomed visualization of the region of interest and of the corresponding regions of interest in the available or selected scans.

In general, to enable real-time generation of the focus views, it may be desirable to estimate the dense displacement fields between the reference scan and each of the other scans, and to store these dense displacement fields in memory so as to avoid having to re-estimate the dense displacement fields in response to a selection of another region of interest. As the generation of the focus views does not necessitate sub-pixel or sub-voxel accuracy, the dense displacement field(s) may be estimated, or converted into a format having integer precision. As displacements are typically not large, a representation of, e.g., 8 bit per coordinate may in certain cases be sufficient to store the displacement. The storage requirement may be further reduced by only estimating, and/or subsequently storing, the dense displacement field(s) in a coarser resolution, e.g., lower than originally estimated and/or lower than the spatial resolution of the scan. A spatial interpolation may then be performed ‘on the fly’ when using the dense displacement field(s). It will be appreciated that in addition to translating each of the scans with respect to the focus views, the image data shown in the focus view may also be rotated. For example, a rotation may be estimated from the displacement vectors in the region of interest, e.g., as a curl or rotor of the dense displacement field in the region of interest. Furthermore, the vector which is used to identify the corresponding region(s) of interest may be a vector which is centrally located in the region of interest, e.g., the geometric center or a weighted center. Alternatively, several vectors may be selected and filtered to obtain a single vector which may then be used. For example, a mean or median of the vectors within the region of interest may be calculated. Alternatively, if the user selects the region of interest by selecting a single point, e.g., a point of interest, the vector located at or nearest to the point of interest may be used.

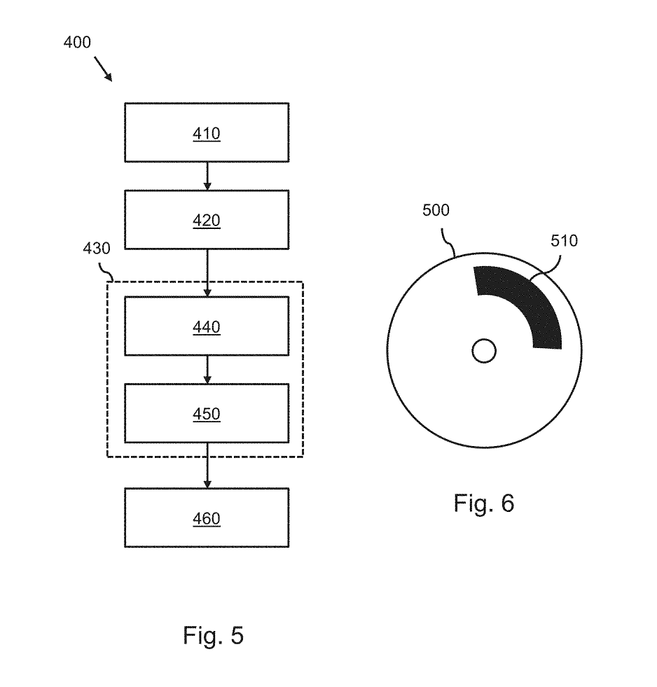

FIG. 5 shows a method 400 for displaying medical images. It is noted that the method 400 may, but does not need to, correspond to an operation of the system 100 as described with reference to FIG. 1 and others. The method 400 may comprise, in an operation titled “ACCESSING MEDICAL IMAGES”, accessing 410 a database comprising a first medical image and a second medical image. The method 400 may further comprise, in an operation titled “RECEIVING SELECTION DATA INDICATIVE OF REGION OF INTEREST”, receiving 420 selection data indicative of a region of interest in the first medical image. The method 400 may further comprise, in an operation titled “IDENTIFYING CORRESPONDING REGION OF INTEREST”, identifying 430 a corresponding region of interest in the second medical image, which may comprise, in an operation titled “ESTIMATING DISPLACEMENT FIELD”, estimating 440 a displacement field by performing a non-linear registration between the first medical image and the second medical image, and in an operation titled “IDENTIFY CORRESPONDING REGION OF INTEREST USING DISPLACEMENT VECTOR(S)”, identifying 450 the corresponding region of interest using one or more displacement vectors of the displacement field which match the region of interest in the first medical image to the corresponding region of interest in the second medical image. The method 400 may further comprise, in an operation titled “GENERATING OUTPUT IMAGE”, generating 460 display data comprising a first viewport and a second viewport, the first viewport comprising a part of the first medical image which shows the region of interest, the second viewport comprising a part of the second medical image which shows the corresponding region of interest. It will be appreciated that the above operation may be performed in any suitable order, e.g., consecutively, simultaneously, or a combination thereof, subject to, where applicable, a particular order being necessitated, e.g., by input/output relations.

The method 400 may be implemented on a computer as a computer implemented method, as dedicated hardware, or as a combination of both. As also illustrated in FIG. 6, instructions for the computer, e.g., executable code, may be stored on a computer readable medium 500, e.g., in the form of a series 510 of machine readable physical marks and/or as a series of elements having different electrical, e.g., magnetic, or optical properties or values. The executable code may be stored in a transitory or non-transitory manner. Examples of computer readable mediums include memory devices, optical storage devices, integrated circuits, servers, online software, etc. FIG. 6 shows an optical disc 500. It will be appreciated that the described system and method may be advantageously applied in the following context, but are not limited to this context.

A major challenge for the analysis of longitudinal data is to determine corresponding locations in all scans. For example, in lung or breast cancer screening, guiding the user to the same anatomical position in all scans helps to easily assess the growth of specific structures. In other applications, the same technique can facilitate monitoring and evaluating the success of treatments. Establishing correspondences may be achieved by image registration techniques, which yield a transformation that maps image coordinates of one image to anatomically corresponding coordinates in another image. However, the optimal way of visualizing aligned scans under consideration of the registration result remains a challenge. A common way of visualizing aligned scans is to use the transformation obtained by image registration to warp all images to a common coordinate system (usually the coordinate system of a chosen reference image). In this way, a given image coordinate may always corresponds to the same anatomical location in all scans. However, deforming an image with the transformation is ill-suited when it is desired to assess changes in pathologies, for example, the growth of lung nodules. The described system and method may address this problem by providing a synchronized display without distorting the image content.

Examples, embodiments or optional features, whether indicated as non-limiting or not, are not to be understood as limiting the invention as claimed.

It will be appreciated that the invention also applies to computer programs, particularly computer programs on or in a carrier, adapted to put the invention into practice. The program may be in the form of a source code, an object code, a code intermediate source and an object code such as in a partially compiled form, or in any other form suitable for use in the implementation of the method according to the invention. It will also be appreciated that such a program may have many different architectural designs. For example, a program code implementing the functionality of the method or system according to the invention may be sub-divided into one or more sub-routines. Many different ways of distributing the functionality among these sub-routines will be apparent to the skilled person. The sub-routines may be stored together in one executable file to form a self-contained program. Such an executable file may comprise computer-executable instructions, for example, processor instructions and/or interpreter instructions (e.g. Java interpreter instructions). Alternatively, one or more or all of the sub-routines may be stored in at least one external library file and linked with a main program either statically or dynamically, e.g. at run-time. The main program contains at least one call to at least one of the sub-routines. The sub-routines may also comprise function calls to each other. An embodiment relating to a computer program product comprises computer-executable instructions corresponding to each processing stage of at least one of the methods set forth herein. These instructions may be sub-divided into sub-routines and/or stored in one or more files that may be linked statically or dynamically. Another embodiment relating to a computer program product comprises computer-executable instructions corresponding to each means of at least one of the systems and/or products set forth herein. These instructions may be sub-divided into sub-routines and/or stored in one or more files that may be linked statically or dynamically.

The carrier of a computer program may be any entity or device capable of carrying the program. For example, the carrier may include a data storage, such as a ROM, for example, a CD ROM or a semiconductor ROM, or a magnetic recording medium, for example, a hard disk. Furthermore, the carrier may be a transmissible carrier such as an electric or optical signal, which may be conveyed via electric or optical cable or by radio or other means. When the program is embodied in such a signal, the carrier may be constituted by such a cable or other device or means. Alternatively, the carrier may be an integrated circuit in which the program is embedded, the integrated circuit being adapted to perform, or used in the performance of, the relevant method.

It should be noted that the above-mentioned embodiments illustrate rather than limit the invention, and that those skilled in the art will be able to design many alternative embodiments without departing from the scope of the appended claims. In the claims, any reference signs placed between parentheses shall not be construed as limiting the claim. Use of the verb “comprise” and its conjugations does not exclude the presence of elements or stages other than those stated in a claim. The article “a” or “an” preceding an element does not exclude the presence of a plurality of such elements. The invention may be implemented by means of hardware comprising several distinct elements, and by means of a suitably programmed computer. In the device claim enumerating several means, several of these means may be embodied by one and the same item of hardware. The mere fact that certain measures are recited in mutually different dependent claims does not indicate that a combination of these measures cannot be used to advantage.

1. A system (100) for displaying medical images, comprising:
an image data interface (120) configured to access image data of a first medical image (022) and a second medical image (024);
a memory (130) comprising instruction data representing a set of instructions;
a processor (160) configured to communicate with the image data interface and the memory and to execute the set of instructions, wherein the set of instructions, when executed by the processor, configure the processor to:
receive selection data (042) indicative of a region of interest (220-224) in the first medical image;
generate display data (062) comprising a first viewport (314), the first viewport comprising a part (200) of the first medical image which shows the region of interest;
identify a corresponding region of interest in the second medical image; and
generate the display data to additionally comprise a second viewport (310, 312), the second viewport comprising a part (202) of the second medical image which shows the corresponding region of interest;
wherein the set of instructions, when executed by the processor, configure the processor to identify the corresponding region of interest in the second medical image by:
estimating a displacement field (230) by performing a non-linear registration between the first medical image and the second medical image; and
identifying the corresponding region of interest using one or more displacement vectors (232) of the displacement field which match the region of interest in the first medical image to the corresponding region of interest in the second medical image.

2. The system (100) according to claim 1, wherein the set of instructions, when executed by the processor (160), configure the processor to apply a spatial interpolation to the displacement field (230) to determine the one or more displacement vectors (232) which match the region of interest in the first medical image (022) to the corresponding region of interest in the second medical image (024).

3. The system (100) according to claim 1 or 2, wherein the set of instructions, when executed by the processor (160), configure the processor to estimate or convert the displacement field (230) in a format having at least one of:
a vector precision limited to integer precision; and
a spatial resolution which is lower than the spatial resolution of the first medical image (022) and/or the second medical image (024).

4. The system (100) according to claim 1, wherein the set of instructions, when executed by the processor (160), configure the processor to re-use the displacement (230) field to identify another corresponding region of interest in the second medical image (024) in response to subsequently received selection data (042) which is indicative of another region of interest in the first medical image (022).

5. The system (100) according to any one of claims 1 to 4, further comprising a user input interface (140) connectable to a user input device (040) operable by a user, wherein the selection data (042) represents a selection of the region of interest using the user input device.

6. The system (100) according to claim 5, wherein the set of instructions, when executed by the processor (160), configure the processor to generate the display data (062) to additionally comprise a further viewport (320) which shows the first medical image, and wherein the selection data (042) represents a selection of the region of interest in the third viewport using an onscreen pointer controllable by the user input device.

7. The system (100) according to claim 5, wherein the set of instructions, when executed by the processor (160), configure the processor to generate the display data (062) to additionally comprise a further viewport which shows a list of regions of interest comprised in the first medical image, and wherein the selection data (042) represents a selection of the region of interest from said list.

8. The system (100) according to any one of claims 1 to 7, wherein the set of instructions, when executed by the processor (160), configure the processor to:
estimate a rotation between the first medical image (022) and the second medical image (024) from the displacement field (230); and
rotate the part (202) of the second medical image to compensate for the rotation before showing said part in the second viewport.

9. The system (100) according to claim 8, wherein the set of instructions, when executed by the processor (160), configure the processor to estimate the rotation from the displacement field (230) in, or in a neighborhood of, a region of the displacement field which corresponds to the region of interest in the first medical image.

10. The system (100) according to any one of the above claims, wherein the first medical image (022) and the second medical image (024) represent longitudinal imaging data

11. A server, workstation or imaging apparatus comprising the system according to any one of claims 1 to 10.

12. A method (400) for displaying medical images, comprising:
accessing (410) a database comprising a first medical image and a second medical image;
receiving (420) selection data indicative of a region of interest in the first medical image;
generating (460) display data comprising a first viewport, the first viewport comprising a part of the first medical image which shows the region of interest;
identifying (430) a corresponding region of interest in the second medical image; and
generating (460) the display data to additionally comprise a second viewport, the second viewport comprising a part of the second medical image which shows the corresponding region of interest;
wherein the identifying the corresponding region of interest in the second medical image comprises:
estimating (440) a displacement field by performing a non-linear registration between the first medical image and the second medical image; and
identifying (450) the corresponding region of interest using one or more displacement vectors of the displacement field which match the region of interest in the first medical image to the corresponding region of interest in the second medical image.

13. A computer readable medium (500) comprising transitory or non-transitory data (510) representing instructions to cause a processor system to perform the method of claim 12.
